(19) United States
(12) Patent Application Publication
(10) Pub. No.: US 2009/0192504 A1
(43) Pub. Date: Jul. 30, 2009

(54) CRYOSURGERY SYSTEM HAVING UNINTEGRATED DELIVERY AND VISUALIZATION APPARATUS

(75) Inventor: Timothy E. Askew, Baltimore, MD (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: CSA Medical, Inc., Baltimore, MD (US)

(21) Appl. No.: 12/022,013

(22) Filed: Jan. 29, 2008

(51) Int. Cl. A61B 18/02

(52) U.S. Cl. 606/21

(57) ABSTRACT

Systems and methods for delivering cryosurgical treatment. The cryosurgery system comprises a cryogen delivery apparatus configured to deliver a spray of cryogen to target tissue of a patient and an indirect visualization apparatus configured to provide indirect visualization of the target tissue during the cryogen delivery. The indirect visualization apparatus and the cryogen delivery apparatus are constructed and arranged to be operationally unintegrated and physically spaced with respect to each other during the delivery of the cryogen.

### BACKGROUND

1. Field of the Invention

The present invention relates generally to cryosurgery systems, and more particularly, to a cryosurgery system having unintegrated delivery and visualization apparatus.

2. Related Art

A variety of medical conditions are preferentially treated by ablation of tissue within the body. Tissue ablation refers to the removal or destruction of tissue, or of tissue functions. Traditionally, invasive surgical procedures were required to perform tissue ablation. These surgical procedures required the cutting and/or destruction of tissue positioned between the exterior of the body and the site where the ablation treatment was conducted, referred to as the treatment site. Such conventional surgical procedures were slow, costly, high risk, and resulted in a long recovery time.

As such, less invasive procedures have been developed in order to improve the cost-effectiveness and safety of tissue ablation. The conventional less invasive procedures result in the destruction of selected tissues via a probe which penetrates to the ablation treatment site, and which destroys the selected tissue by transferring energy to the tissue. For example, RF energy, light (laser) energy, microwave energy, and high-frequency ultra-sound energy are among the forms of energy which have been used for tissue ablation. However all of these methods have the common disadvantage that while energy is transferred to the desired tissue, energy is also inadvertently transferred, through conduction, convection, or some other natural processes, to nearby healthy tissue(s) as well. Furthermore, the energy transfer results in heat release, causing surgical complications and potential adverse effects, including noticeable pain, functional impairment of nearby healthy tissue(s), and/or damage or destruction of nearby healthy tissue(s). Moreover, in some cases, the exposure of tissue to thermal energy or other forms of energy may raise the tissue's temperature, thereby causing the tissue to secrete substances that may be toxic to adjacent healthy tissue(s).

In contrast, cryoablation is a procedure in which tissue ablation is conducted by freezing diseased, damaged or otherwise unwanted tissue (collectively referred to herein as “target tissue”). Appropriate target tissue may include, for example, cancerous or precancerous lesions, tumors (malignant or benign), fibroses and any other healthy or diseased tissue for which cryoablation is desired.

Typically, cryoablation procedures are carried out through the use a solid probe that has been cooled to a low temperature. In such a procedure, the low temperature probe is placed in contact with a diseased or damaged portion of tissue, thereby causing the target tissue to freeze. Recently it has been discovered that cryoablation may also be performed by using a system that sprays low pressure cryogen on the target tissue. Such systems are referred to as cryosurgery spray systems, or simply, cryosurgery systems, herein. Also as used herein, cryogen refers to any fluid (e.g., gas, liquefied gas or other fluid known to one of ordinary skill in the art) that has a sufficiently low boiling point to allow for therapeutically effective cryotherapy and is otherwise suitable for cryogenic surgical procedures. For example, acceptable fluids may have a boiling point below approximately negative (−) 150° C. The cryogen may be nitrogen, as it is readily available. Other fluids such as argon and air may also be used.

During operation of a cryosurgery system, a clinician, physician, surgeon, technician, or other operator, (collectively referred to as “operator” herein) sprays cryogen on the target tissue via a delivery catheter. The spray of cryogen causes the target tissue to freeze or “cyrofrost.” This freezing of the tissue often causes the target tissue to acquire a white color (indicative of cryofrost). The white color indicates that the target tissue has been sufficiently frozen to destroy any diseased tissue. The temperature range for cryofrost can be approximately negative (−) 10° C. to approximately negative (−) 90° C. However, the particular temperature for cryofrost will depend on the target tissue, including size, location, etc. The time period to reach cryofrost may vary, from approximately 5 seconds to approximately 2 minutes or more depending on the size and location of the target tissue and the thermodynamic potential of the cryogen. A cryosurgery system may include a camera system that enables the operator to monitor the cryogen delivery and determine when cyrofost has occurred.

### SUMMARY

According to one aspect of the present invention, there is provided a cryosurgery system comprising: a cryogen delivery apparatus configured to deliver a spray of cryogen to target tissue of a patient; and an indirect visualization apparatus configured to provide indirect visualization of the target tissue during the cryogen delivery, wherein the indirect visualization apparatus and the cryogen delivery apparatus are constructed and arranged to be operationally unintegrated and physically spaced with respect to each other during the delivery of the cryogen.

In another aspect of the present invention, there is provided a method of delivering cryogen to target tissue within a patient via a cryosurgery system, comprising: adjusting the relative physical orientation of the patient and an indirect visualization apparatus; inserting a cryogen delivery apparatus that is physically spaced apart from, and operationally unintegrated from the indirect visualization apparatus into the patient; positioning the cryogen delivery apparatus in the patient without requiring concomitant movement of the indirect visualization apparatus; delivering the cryogen to the target tissue; and monitoring the delivery of the cryogen with the physically spaced apart indirect visualization device.

In a third aspect of the present invention, there is provided a cryogen delivery system comprising: a cryogen delivery means for delivering a spray of cryogen to target tissue of a patient; a viewing means for indirectly viewing the delivery of the cryogen to the target tissue; wherein the delivery means and the viewing means are configured to be operationally unintegrated and physically spaced with respect to each other during the delivery of the cryogen.

### BRIEF DESCRIPTION OF THE DRAWINGS

Embodiments of the present invention will be described in conjunction with the accompanying drawings, in which:

FIG. 1 is a perspective view of one embodiment of an exemplary cryosurgery system in which aspects of the present invention may be implemented;

FIG. 2 is a perspective view of an integrated cryogen delivery system according to the prior art;

FIG. 3 is a representative schematic block diagram illustrating a prior art integrated cryogen delivery system, an example of which is illustrated in FIG. 2;

FIG. 4 is a representative schematic block diagram of the unintegrated cryosurgery system according to embodiments of the present invention;

FIG. 5A is a schematic block diagram of aspects of an unintegrated cryosurgery system according to embodiments of the present invention;

FIG. 5B is a schematic block diagram of alternative aspects of an unintegrated cryosurgery system according to embodiments of the present invention;

FIG. 6 is a flowchart demonstrating the cryosurgery treatment of a patient according to the system of FIG. 4, in accordance with embodiments of the present invention;

FIG. 7 is a perspective view of one embodiment of the unintegrated cryosurgery system of FIG. 5A;

FIG. 8A is a perspective view of one embodiment of the unintegrated cryosurgery system of FIG. 5B; and

FIG. 8B is a perspective view of an additional embodiment of the unintegrated cryosurgery system of FIG. 5B.

### DETAILED DESCRIPTION

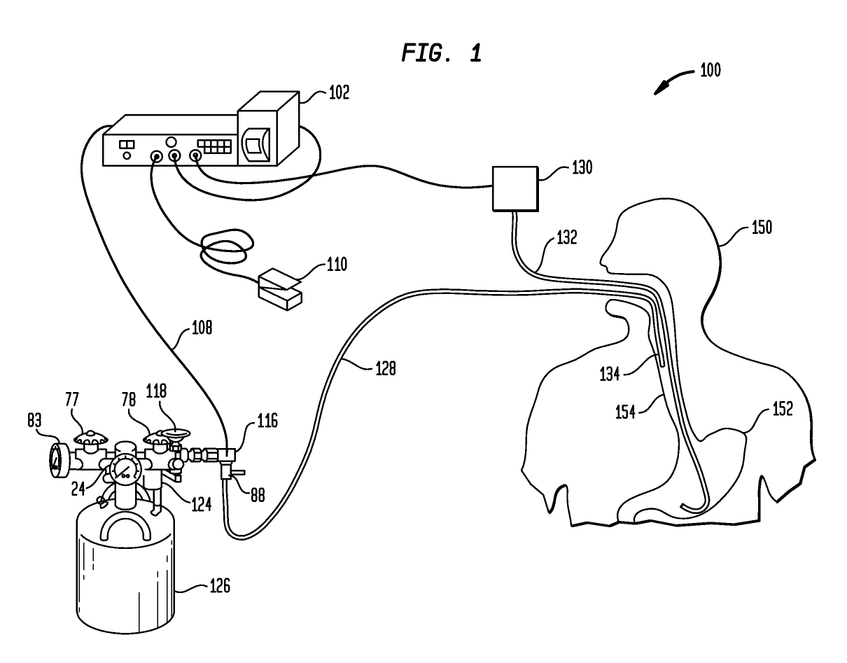

Embodiments of the present invention are directed to a cryosurgery system having a cryogen delivery apparatus that is physically spaced and operationally unintegrated from an indirect visualization system. In accordance with embodiments of the present invention, the cryosurgery system may further include a cryogen source configured to provide the cryogen to the cryogen delivery apparatus, a regulation apparatus fluidically coupled to the cryogen source and to the cryogen delivery apparatus, and a controller communicatively coupled to the regulation apparatus configured to control the release of cryogen into the cryogen delivery apparatus. Exemplary cryosurgery systems in which the present invention may be implemented include, but are not limited to, those systems described in commonly owned U.S. Pat. Nos. 7,025,762, 6,383,181, 6,027,499 and U.S. patent application Ser. No. 10/352,266, which are hereby incorporated by reference herein. Embodiments of the present invention are described below in connection with one embodiment of such exemplary cryosurgery system shown in FIG. 1.

Utilization of an unintegrated indirect visualization system and cryogen delivery apparatus is particularly beneficial because it allows for independent positioning and/or guiding of the cryogen delivery apparatus with respect to the indirect visualization system. Such independent positioning and/or guiding provides the operator with a wide variety of positions, angles, etc. from which to deliver the cryogen while simultaneously having the freedom to independently adjust the physical location, orientation, viewing angle, and/or other aspect of the visualization system. Advantageously, this may provide the patient with potentially less invasive treatment options. Furthermore, the dimensions of the cryogen delivery apparatus may be beneficially reduced in size to fit into smaller pathways or cavities. As such, a larger variety of target tissue may be accessed by the operator. Furthermore, a smaller size cryogen delivery apparatus permits the insertion of the cryogen delivery apparatus into a greater number of access points with less trauma.

A simplified perspective view of an exemplary cryosurgery system in which embodiments of the present invention may be implemented is illustrated in FIG. 1. Cryosurgery system 100 comprises a pressurized cryogen storage tank 126 to store cryogen under pressure. In the following description, the cryogen stored in tank 126 is liquid nitrogen although cryogen may be other materials as described in detail below. A convenient size for tank 126 has been found to be a 5.5 liter size, although larger or smaller size tanks may be implemented depending on the particular application and operational environment. In one embodiment, tank 126 is a double-walled insulated tank with adequate insulation to maintain the liquid nitrogen at a very low temperature over a long period of time. In one embodiment, the pressure for the liquefied gas in tank is 22 psi. However, it is to be understood that tank 126 may maintain the liquid nitrogen or other cryogen at other pressures suitable for the particular application.

Tank 126 is equipped with a pressure building coil or tube 124 for maintaining pressure. This tube 124 comprises metal tubing running from the inside of tank 126 to the outside of tank 126 and returning back to the inside of tank 126. Tube 124, in operation, contains circulating liquid nitrogen. If the pressure in tank 126 drops below acceptable levels, valve 118 to tube 124 may be opened to circulate gas outside of tank 126 through tube 124. The liquid nitrogen in tube 124 outside tank 126 will be warmed and returned to tank 126. This warmed nitrogen liquid will cause the head pressure in tank 126 to increase, thereby allowing for more rapid delivery of liquid nitrogen to a cryogen delivery catheter 128. In the tube arrangement shown, valve 118 is hand-operated, however, valve 118 could be automatically controlled. In such an embodiment, valve 118 may be controlled to start circulating liquid through tube 124 or a coil once the pressure in tank 126 drops to unacceptable levels, and to stop circulating once the pressure returns to an acceptable level. With normal pressure maintained in tank 126, liquefied gas will be more rapidly expelled from tank 126 to catheter 128. The force of gas expelled from tank 126 is a function of the temperature and pressure of the liquid nitrogen in tank 126. Because of the large temperature differential between the ambient temperature and the temperature of liquid nitrogen, only a short length of tube 124 is required.

Tank 126 is also equipped with other valves and gauges. A head gas valve 77 relieves head pressure, while a delivery solenoid valve 78 allows liquid nitrogen to flow to catheter 128 through controllable valve 1 16. Safety relief valves (not shown) on tank 126 are configured to relieve tank 126 of excessive tank pressure. For example, in one embodiment, two safety relief valves are implemented; one valve may open at 22 psi and the other valve may open at 35 psi. In addition, tank 126 is equipped with a head pressure gauge 83 and a liquid level gauge 84.

In this exemplary cryosurgery system, a foot pedal 110 is implemented to allow operator actuation of controllable valve 116. Foot pedal 110 has the advantage of allowing the physician's hands to be free during cryosurgery. Tank 126, heating tube 124, and foot pedal 110 collectively allow for quick delivery of adequate amounts for cryogenic spray to tissue requiring cryoablation.

In certain embodiments, cryosurgery system 100 forces super-cooled nitrogen gas through catheter 128 at low pressure. This is accomplished with an auxiliary pressure bleeder 88 positioned between tank 126 and catheter 128. Bleeder 88 eliminates the elevated pressure produced at catheter 128 caused by the reduced internal diameter of catheter 128 relative to the larger internal diameter of the tube supplying nitrogen gas to catheter 128; and by the volatilization of the liquid nitrogen to gas phase nitrogen. Bleeder 88 reduces such pressure by venting gas phase nitrogen out of bleeder 88. With this venting of gas phase nitrogen, liquid phase nitrogen exits the distal end of catheter 128 as a mist or spray at a pressure of approximately 35 psi compared with the tank pressure of approximately 22 psi. It is to be understood that bleeder 88 is used in this exemplary embodiment, but that other embodiments of the cryosurgery system do not require bleeder 88.

In the embodiment illustrated in FIG. 1, a conventional therapeutic endoscope 134 is used to deliver the nitrogen gas to target tissue within the patient. Endoscope 134 may be of any size, although a smaller diagnostic endoscope is preferably used from the standpoint of patient comfort. In certain embodiments, a specially designed endoscope having a camera integrated therein may also be used. As is known, an image received at the lens on the distal end of the camera integrated into endoscope 134 may be transferred via fiber optics to a monitoring camera which sends video signals via a cable to a conventional monitor or microscope, where the procedure can be visualized. By virtue of this visualization, the surgeon is able to perform the cryosurgery at treatment site 154.

As the liquid nitrogen travels from tank 126 to the proximal end of cryogen delivery catheter 128, the liquid is warmed and starts to boil, resulting in cool gas emerging from the distal end or tip of catheter 128. The amount of boiling in catheter 128 depends on the mass and thermal capacity of catheter 128. Since catheter 128 is of small diameter and mass, the amount of boiling is not great. (The catheter would preferably be “French Seven”.) When the liquid nitrogen undergoes phase change from liquid to gaseous nitrogen, additional pressure is created throughout the length of catheter 128. This is especially true at the solenoid/catheter junction, where the diameter of the supply tube relative to the lumen of catheter 128 decreases from approximately 0.5 inches to approximately 0.062 inches, respectively. In order to force low pressure liquid/gas nitrogen through this narrow opening, either the pressure of the supplied nitrogen must decrease or the diameter of catheter 128 must increase. Due to the fact that system 100 is not a highly pressurized system, a bleeder 88 may be implemented to solve this problem. Bleeder 88 is configured to allow the liquid phase nitrogen to pass through the reduced diameter catheter 128 without requiring modification of tank pressure or catheter diameter. Without a pressure bleeder 88, the pressure of gas leaving the distal end of catheter 128 would be too high and have the potential for injuring the tissue of the patient.

When the liquid nitrogen reaches the distal end of catheter 128 it is sprayed out of cryogen delivery catheter 128 onto the target tissue. It should be appreciated that certain embodiments the cryosurgery system may be able to sufficiently freeze the target tissue without actual liquid nitrogen being sprayed from catheter 128. In particular, a spray of liquid may not be needed if cold nitrogen gas is capable of freezing the target tissue.

Freezing of the target tissue is apparent to the physician by the acquisition of a white color, referred to as cryofrost, by the target tissue. The white color, resulting from surface frost, indicates mucosal freezing sufficient to destroy the diseased tissue. In one embodiment, the composition of catheter 128 or the degree of insulating capacity thereof will be selected so as to allow the freezing of the mucosal tissue to be slow enough to allow the physician to observe the degree of freezing and to stop the spray as soon as the surface achieves the desired whiteness of color. The operator may monitor the target tissue to determine when cryofrost has occurred via the camera integrated into endoscope 134. The operator manipulates suction tube 132 and/or cryogen catheter 128 to freeze the target tissue. Once the operation is complete, tube 132, catheter 128, and endoscope 134 are withdrawn.

Because the invention uses liquid spray via catheter 128 rather than contact with a cold solid probe, the risk that an apparatus may stick to the tissue of the patient is reduced. Catheter 128 is further constructed and arranged so to reduce the potential for damage to the patient's tissue during the cryosurgery. For example, catheter 128 may comprise a plastic material having a low thermal conductivity and specific heat transfer properties, such as TEFLON®, that reduces the potential that catheter 128 may stick to the tissue of the patient

Using cryogen delivery catheter 128 to deliver the cryogen permits a higher cooling rate (rate of heat removal) since the sprayed liquid evaporates directly on the tissue to which the cryogen is applied. The rate of re-warming of the target tissue is also high due to the fact that the applied liquid nitrogen boils away rapidly. No cold liquid or solid remains in contact with the tissue, and the depth of freezing is minimal.

Treatment site 154 as depicted in FIG. 1 is the esophagus of patient 150. It should be appreciated, however, that the treatment site but may be any location within patient 150 such as inside stomach 152 or other cavities, crevices, vessels, etc. Since freezing is accomplished by boiling liquid nitrogen, large volumes of this gas are generated. This gas must be allowed to escape. The local pressure will be higher than atmospheric pressure since the gas cannot easily flow out of the treatment site such as the gastrointestinal tract. In the illustrated embodiment, nitrogen gas will tend to enter stomach 152, which has a junction with the esophagus (the esophageal sphincter) immediately adjacent to treatment site 154. In this case, without adequate or quick suction, stomach 152 of patient 150 may become distended and become uncomfortable for patient 150. This buildup of gas could also potentially cause stomach 152 or its lining to become damaged or torn. As such, to prevent this buildup of gas in stomach 152, a suction tube 132 (e.g., a nasogastric tube) may be inserted into the patient to evacuate cryogen and other gases, particles, liquids, etc. from the patient. Suction may be provided by a suction pump 130 or other conventional source of negative pressure.

Also depicted in FIG. 1 is a control unit 102, which is connected to foot pedal 110, controllable valve 116 and pump 130. In this embodiment, an operator of cryosurgery system 100 may instruct control unit 102 to actuate controllable valve 116 via foot pedal 110. The operator may start the flow of cryogen by pressing on foot pedal 110, and may end the flow of cryogen by releasing foot pedal 110. The flow of cryogen may be fluctuated by exerting differing amounts of pressure on foot pedal 110. Actuation of foot pedal 110 causes control unit 102 controls controllable valve 116 via control line 108 to cause controllable valve 116 to open or close based on, for example, receiving operator inputs, thermal sensors (not shown) located at one or more points in system 100 or the environment outside system 100, pressure sensors (not shown), among others inputs. Although this illustrative embodiment describes the use of foot pedal 110 to enter user inputs it should be appreciated that other manners of entering operator inputs may be utilized, including buttons, switches, toggles, dials, user interfaces, etc. on, in, or coupled to control unit 102.

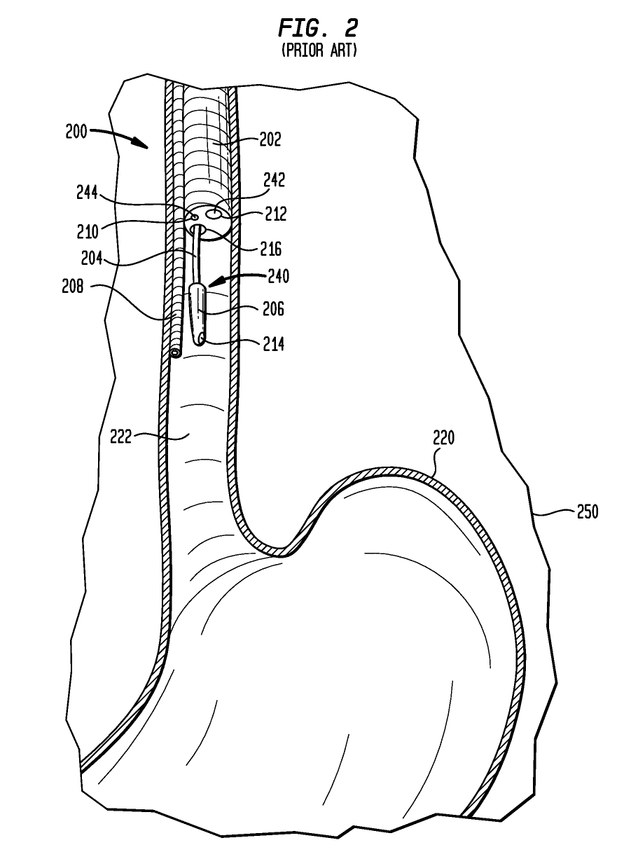

FIG. 2 is a perspective view of a portion of a prior art integrated cryosurgery system 200 having a cryogen delivery apparatus 240 physically integrated with an endoscope camera 242. Integrated cryosurgery system 200 comprises an endoscope 202 having lumens 210, 212 and 216 therein. As shown, endoscope 200 may be positioned in the esophagus 222 of patient 250. Lumen 212, disposed in endoscope 202, is configured to receive an endoscope camera 242. An image received at the lens of endoscope camera 242 may be transferred via fiber optics to a monitoring camera. The monitoring camera then sends video signals via a cable to a conventional monitor or microscope, where the image captured by the lens can be visualized. As shown in FIG. 2, endoscope camera 242 may be inserted through lumen 212 to allow an operator to view the cryosurgery procedure. Lumen 210 is configured to have disposed therein a light 244 that is configured to illuminate the treatment site.

Lumen 216 is configured to receive cryogen delivery apparatus 240. Cryogen delivery apparatus 240 comprises a cryogen delivery catheter 204, catheter tip 206, and one or more holes 214. After insertion of the cryogen delivery apparatus into the patient, cryogen is provided to cryogen delivery catheter 204 from a cryogen source (not shown). Tip 206 causes the cryogen to be sprayed on the target tissue via hole 214. A suction tube 208 is provided to evacuate the treatment area of undesirable gases, particles, fluids etc.

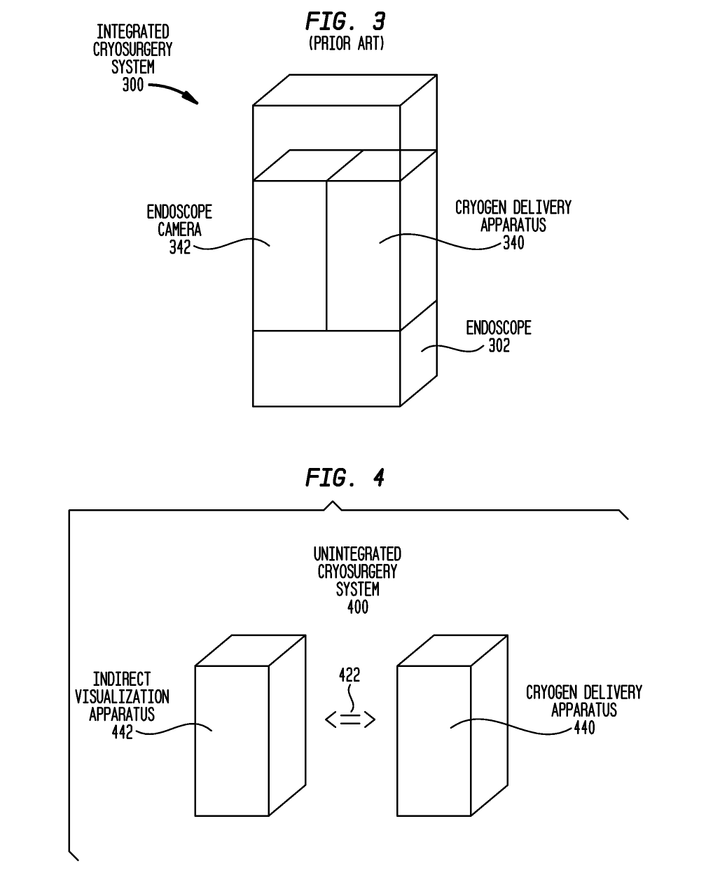

FIG. 3 is schematic block diagram representing the integrated physical relationship of cryogen delivery apparatus 340 and an endoscope camera 342 in endoscope 302. For example, with reference to cryosurgery system 200 of FIG. 2, block 302 is a schematic representation of endoscope 202. Block 340 is a representation of cryogen delivery apparatus 240. Block 342 is a representation of endoscope camera 342. Blocks 340 and 342 are shown integrated within block 302. FIG. 3 schematically demonstrates that in the convention cryosurgery system 200, endoscope camera 242 and cryogen delivery apparatus 240 are, during operation, integrated within endoscope 202.

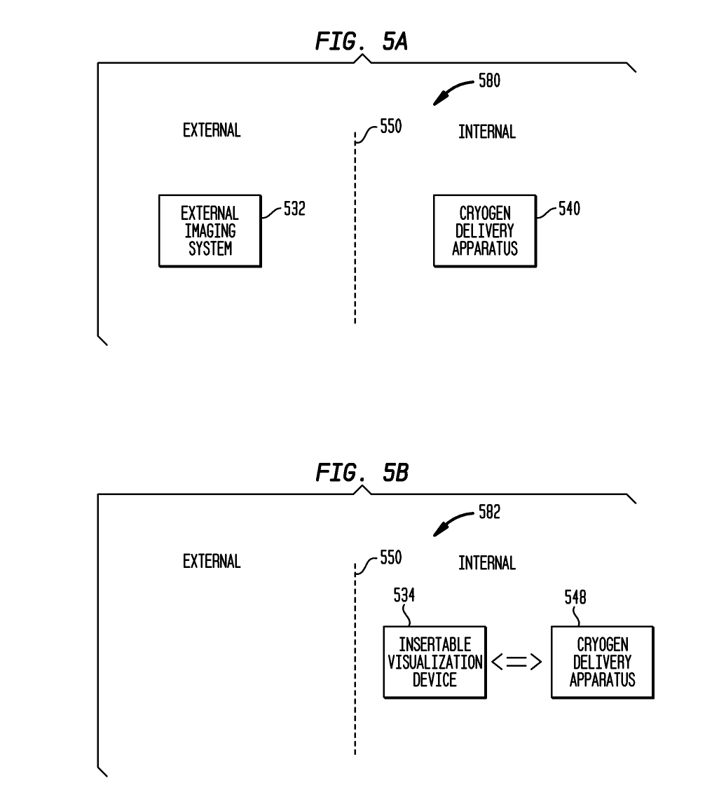

In contrast to FIG. 3, FIG. 4 is a schematic view of a cryosurgery system according to embodiments of the present invention. As shown in FIG. 4, unintegrated cryosurgery system 400 comprises blocks 440 and 442. Block 440 is a representation of a cryogen delivery apparatus configured to deliver cryogen to target tissue. Block 442 is a representation of an indirect visualization apparatus according to embodiments of the present invention that permit an operator to view the delivery of cryogen. The indirect visualization device can be further utilized to assist in the insertion of the cryogen delivery apparatus. Unlike blocks 340 and 342 from FIG. 3, blocks 440 and 442 are shown physically spaced apart by spacing arrow 422. The spacing of blocks 440 and 442 illustrates that in embodiments of the present invention the utilized indirect visualization apparatus and cryogen delivery apparatus are unintegrated and physically spaced apart with respect to one another. Unintegrated refers to the indirect visualization apparatus and cryogen delivery apparatus being physically independent and spaced-apart components. In other words, the indirect visualization apparatus and cryogen delivery apparatus are neither integrated, taken into, nor made a part of a unitary structure, such as an endoscope of the prior art device of FIG. 3.

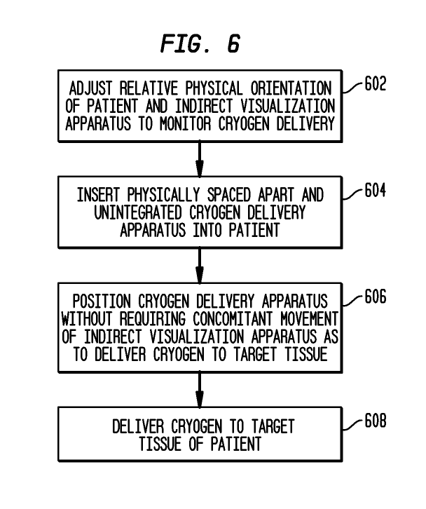

FIG. 5A is a schematic block diagram illustrating embodiments of an unintegrated cryosurgery system 580 having an embodiment of cryogen delivery apparatus 440, referred to as cryogen delivery apparatus 540, and an embodiment of an indirect visualization apparatus 442, referred to as external imaging system 532. Cryogen delivery apparatus 540 and external imaging system 532 are constructed and arranged to be operationally unintegrated and physically spaced with respect to each other. These components are shown relative to a patient, schematically represented by line 550. In the embodiments of FIG. 5A, cryogen delivery apparatus 540 is positioned within patient 550, while external imaging system 532 is positioned external to patient 550.

External imaging system 532 may be any device positioned external to patient 550 that allows the operator to view and/or monitor the delivery of cryogen to the target tissue. In certain embodiments, external imaging system 532 is further configured to assist the operator in inserting cryogen delivery apparatus 540 into patient 550. Such devices are generally referred to as medical imaging devices. Medical imaging devices provide the operator with a visual representation of space, tissue, etc. within patient 550. The visual representation is not limited only to images captured through the use of an imaging device, but the visual representation may also include representations derived from data captured from one or multiple devices operating in one or more data capturing modes.

Various types of such medical imaging devices may be advantageously utilized in the present invention. For example, conventional medical imaging devices such as a Computed Tomography (CT) system, an ultrasound scanning system, a X-ray system, or a Magnetic Resonance Imaging (MRI) system, may be utilized in various embodiments of the present invention. Furthermore, alternative medical imaging devices such as a Gallium scanner, a Digital Subtracted Angiography (DSA) scanner, a Fluoroscopy Imaging system, a positron emission tomography (PET) system, an Optoacoustic Imaging (Photoacoustic Imaging) system, an Electrical Impedance Tomography (EIT) device, etc., may be utilized in various embodiments of the present invention. As would be appreciated, the present invention is not limited to the above examples and may include any other appropriate medical imaging device now known or later developed. The selection of the medical imaging device may depend on a variety of factors, including the type of target tissue (e.g. soft tissue, hard tissue, cartilage), the location of the target tissue, as well as various other aspects of the cryosurgery treatment, etc.

Cryogen delivery apparatus 540 is configured to deliver a spray of cryogen to target tissue within patient 550. In preferred embodiments, cryogen delivery apparatus 540 may comprise a delivery catheter such as that described above with reference to FIG. 1. In further embodiments, cryogen delivery apparatus 540 may comprise, along with a delivery catheter, a catheter tip disposed on the distal end of the delivery catheter. In such embodiments, the catheter tip may be similar to tip 206 described with reference to FIG. 2. Other embodiments of the catheter tip may also be envisioned.

FIG. 5B is a schematic block diagram illustrating embodiments of an unintegrated cryosurgery system 582 having an embodiment of cryogen delivery apparatus 440, referred to as cryogen delivery apparatus 548, and an embodiment of an indirect visualization apparatus 442, referred to as insertable visualization device 534. Cryogen delivery apparatus 548 and insertable visualization device 534 are constructed and arranged to be operationally unintegrated and physically spaced with respect to each other. These components are shown relative to a patient, schematically represented by line 550. In the embodiments of FIG. 5B, cryogen delivery apparatus 548 and insertable visualization device 534 are positioned within patient 550.

Insertable visualization device 534 may be any device that is configured to be at least partially insertable into patient 550. In certain embodiments, insertable visualization device 534 is configured to permit indirect viewing of the cryogen delivery. Insertable visualization device 534 may also be configured to assist in the insertion of cryogen delivery apparatus 548 into patient 550. Furthermore, one insertable visualization device 534 may be used while inserting or otherwise positioning cryogen delivery apparatus 548, while other insertable visualization devices 534 are used during treatment. While the type of device that may utilized as insertable visualization device 534 is not limited, suitable examples include a mirror, such as a dental mirror, an endoscope camera, or a fiber optic cable.

Cryogen delivery apparatus 548 is configured to deliver a spray of cryogen to target tissue within patient 550. In preferred embodiments, cryogen delivery apparatus 548 may comprise a delivery catheter such as that described above with reference to FIG. 1. In further embodiments, cryogen delivery apparatus 548 may comprise, along with a delivery catheter, a catheter tip disposed on the distal end of the delivery catheter. In such embodiments, the catheter tip may be similar to tip 206 described with reference to FIG. 2. Other embodiments of the catheter tip may also be envisioned. It should be appreciated that insertable visualization device 534 may be used, not only to view cryogen delivery apparatus 548, but to view the target tissue or the cryogen being delivered by cryogen delivery apparatus 548.

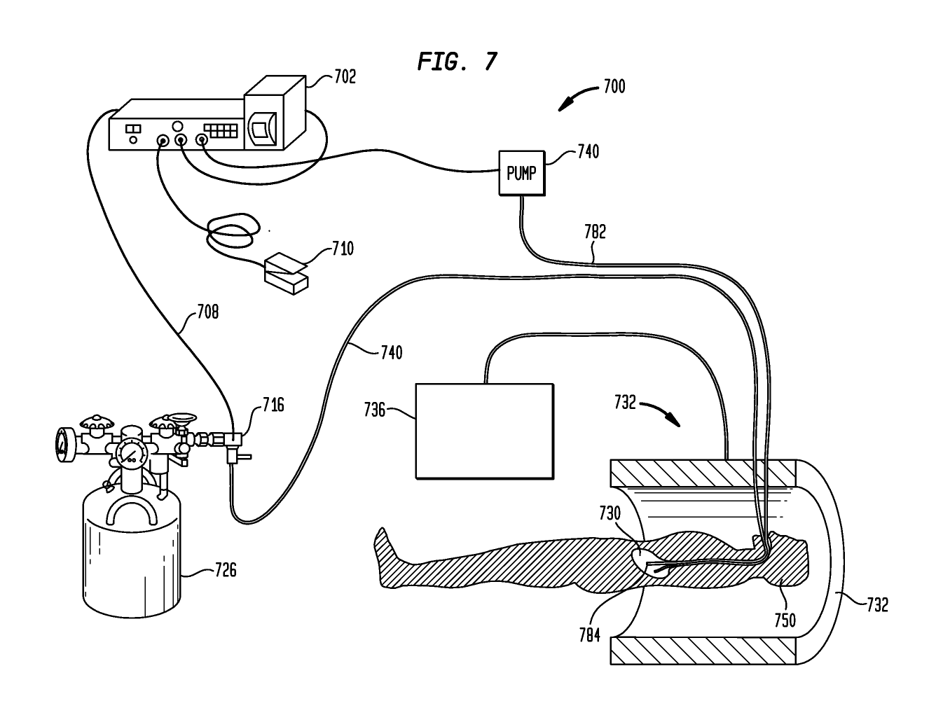

FIG. 6 is a flowchart demonstrating an exemplary cryosurgical procedure utilizing embodiments of unintegrated cryosurgery system 400 of FIG. 4. FIG. 6 includes blocks 602, 604, 606 and 608. At block 602 the operator adjusts the relative physical orientation of patient and indirect visualization apparatus 442 so that the operator is able to indirectly view the cryogen delivery. In certain embodiments, indirect visualization apparatus comprises external imaging system that, as noted above, may comprise a variety of medical imaging devices. Depending on the medical imaging device selected, either the patient or the device may be physically positioned relative to the other so as to monitor the procedure.

In other embodiments, indirect visualization apparatus 442 may comprise an insertable visualization device. In such embodiments, the insertable visualization device is positioned relative to patient by insertion therein.

At block 604 cryogen delivery apparatus 440 that is constructed and arranged to be operationally unintegrated and physically spaced with respect to indirect visualization apparatus 442. At block 606, cryogen delivery apparatus 440 is positioned within the patient so as to deliver cryogen to target tissue. Cryogen delivery apparatus 440 is independently positioned to deliver the cryogen without requiring concomitant movement of indirect visualization apparatus 442.

At block 606 the cryogen is delivered to the target tissue via cryogen delivery apparatus 440. The operator utilizes indirect visualization apparatus 442 to monitor the delivery of cryogen to the target tissue. Indirect visualization apparatus 442 may be further used to determine when cyrofrost has occurred. The cryogen treatment ends at block 608 by removing cryogen delivery apparatus 540 from the patient.

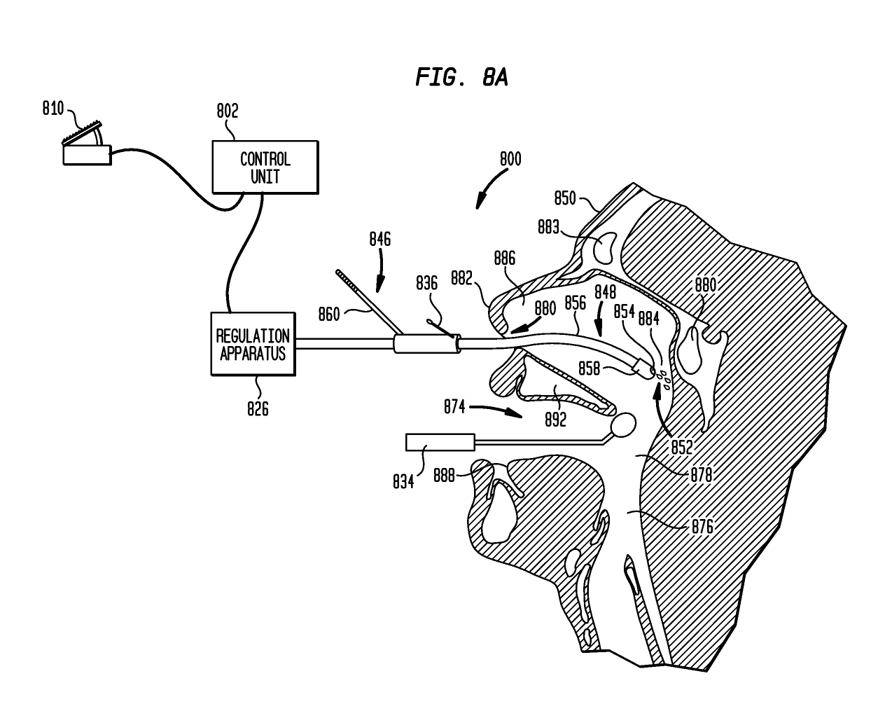

FIG. 7 is a perspective view of one embodiment of unintegrated cryosurgery system 580 of FIG. 5A, referred to as cryosurgery system 700. In this illustrative embodiment, cryosurgery system 700 comprises an external imaging system 732 and a cryogen delivery apparatus 740. As shown, cryosurgery system 700 further comprises a pressurized cryogen storage tank 726, regulation apparatus 716, controller 702, foot pedal 710, vacuum pump 740 and evacuation tube 782.

Cryogen storage tank 726 is configured to store cryogen, such as described with reference to cryogen storage tank 126 of FIG. 1. Cryogen storage tank 726 is fluidically coupled to regulation apparatus 716. Regulation apparatus 716 may comprise any device that is configured to regulate the flow of a fluid or gas there through. For example, regulation apparatus 716 may comprise a valve, regulator, etc., such as controllable valve 116 of FIG. 1. Regulation apparatus 716 is further fluidically coupled to cryogen delivery apparatus 740, the details of which are provided below.

Communicably coupled to regulation apparatus 716 is control unit 702. Control unit 702 is similar to control unit 102 of FIG. 1 and is configured to control the flow of cryogen from cryogen storage tank 726 to cryogen delivery apparatus 740 via regulation apparatus 716. Connected to control unit 702 is foot pedal 710. In the illustrated embodiment, an operator of cryosurgery system 700 may instruct control unit 702 to actuate regulation apparatus 716 via foot pedal 710. Foot pedal 710 is similar to foot pedal 110 of FIG. 1. As previously discussed, the operator may start the flow of cryogen by pressing on foot pedal 710, and may end the flow of cryogen by releasing foot pedal 710. The flow of cryogen may be fluctuated by exerting differing amounts of pressure on foot pedal 710. Although this illustrative embodiment describes the use of foot pedal 710 to enter operator inputs it should be appreciated that other manners of entering operator inputs may be utilized, including buttons, switches, toggles, dials, user interfaces, etc. on, in, or coupled to control unit 702.

As noted above, cryogen flows from storage tank 726 through regulation apparatus 716 to cryogen delivery apparatus 740. In the embodiment illustrated in FIG. 7, cryogen delivery apparatus 740 comprises a delivery catheter 740. As would be appreciated, catheters, such as catheter 740, are tubular, flexible, and have at least one lumen disposed along their elongate body. Delivery catheter 740 may be introduced into the patient via a variety of means including through the use of a stylet, trocar, sheath, etc.

In the embodiment of FIG. 7, an operator positions delivery catheter 740 into patient 750. In the illustrated embodiment delivery catheter 740 is positioned in the stomach 730 of patient 750. Tip 784 of delivery catheter is positioned near the target tissue in order to spray the target tissue with cryogen. The operator then uses foot pedal 710 to start the flow of cryogen. After the target tissue reaches cryofrost, delivery catheter 740 is removed from the patient. It should be appreciated that multiple applications of cryogen spray onto target tissue may be provided during treatment. Between each such application, the target tissue may thaw prior to the next application of cryogen occurs.

As noted above, cryosurgery system 700 also comprises an external imaging system 732 configured to allow the operator of system 700 to view the delivery of cryogen to the target tissue. External imaging system 732 is also configured to optionally permit the operator to view the insertion of cryogen delivery apparatus 740 into patient 750. As noted above with reference to FIG. 5A, external imaging system 732 may comprise a number of various imaging systems including an x-ray system, a computed tomography system, an ultrasound system, or a magnetic resonance imaging (MRI) system. The embodiment of external imaging system 732 illustrated in FIG. 7 comprises an MRI system 732. MRI system 732 comprises an MRI scanner 732 and a screen 736 to view the results of the scan. Although the embodiment of FIG. 7 illustrates the use of an MRI, any of the above-mentioned external imaging systems, or any other systems now known or later developed, may also be utilized.

Illustrative cryosurgery system 700 may further comprise a suction catheter 782 fluidically coupled to vacuum pump 740. During cryosurgery, the cryogen is normally removed from the area near the target tissue to prevent non-target tissue from being exposed to the cryogen's extremely cold temperature or volume. Similarly, other particles, gases or fluids may need to be removed during or after the cryosurgery. This removal may be accomplished via vacuum pump 740 and suction catheter 782 optionally inserted into patient 750.

For ease of explanation, cryogen delivery catheter 740 and external imaging system 732 have been shown inserted into the mouth of patient 750 to provide cryosurgical treatment to target tissue positioned in stomach 730 of patient 750. It should be appreciated that cryogen delivery catheter 740 may be utilized in additional areas or cavities of patient 750, such as the nasal cavity, esophagus, stomach, lung, etc. Similarly, it should be appreciated that in these other embodiments, cryogen delivery catheter 740 may be inserted into patient 750 from different entry points on the body of patient 750. For example, cryogen delivery catheter 740 may be inserted through the nose of patient 750, through a body access interface device such as a trocar, etc. Other entry points and interfaces now known or later developed may also be used in conjunction with other embodiments of the present invention.

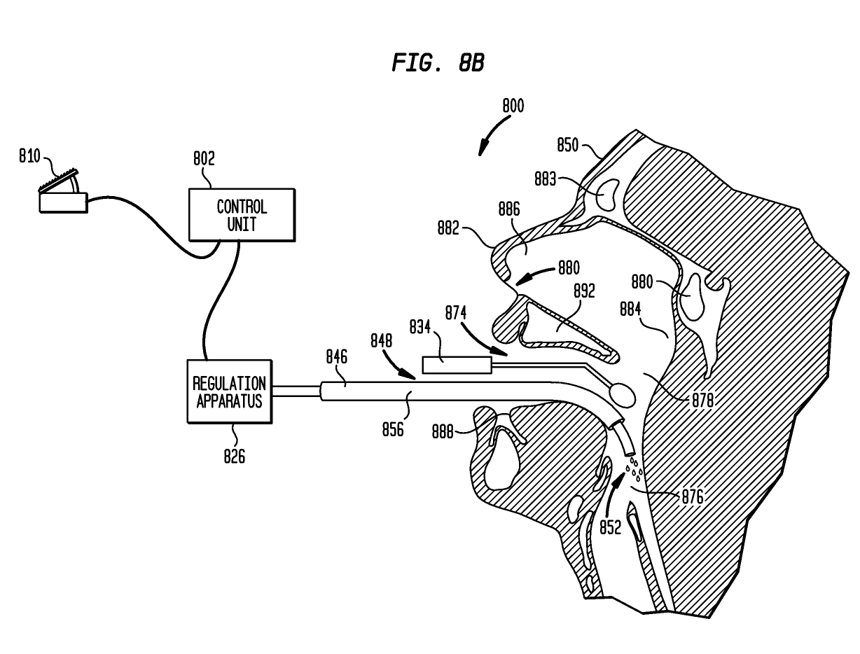

FIG. 8A is a perspective view of one embodiment of unintegrated cryosurgery system 582 of FIG. 5B, referred to as cryosurgery system 800. Cryosurgery system 800 is illustrated in conjunction with a cross-sectional view of the head of a patient 850. For clarity, FIG. 8A illustrates a simplified view of patient 850's head. As such, various structures have been intentionally omitted. For example, the olfactory bulb, nasal conchae, and teeth have been omitted for clarity. However, FIG. 8A does include nasal cavity 886, frontal sinus 883, sphenoid sinus 880, nasopharynx 884, oropharynx 878, tongue 888, esophagus 876, and palate 892 for reference.

Cryosurgery system 800 comprises an insertable visualization device 834 and a cryogen delivery apparatus 848. In this illustrative embodiment, cryosurgery system 800 is configured to provide cryogen to target tissue, for example, on the walls of the nasopharynx 884 or on the walls of nasal cavity 886 of patient 850. Nasopharynx 884 is the portion of the throat that connects the back of nasal cavity 886 to the back of mouth 874. The walls of nasopharynx 884 or of nasal cavity 886 may prove difficult to reach with conventional integrated cryosurgery systems because the entrance to nasal cavity 886 through nose 882, referred to as nostrils 880, may be too narrow to receive an integrated cryosurgery system. As such, the operator may be forced to guide the integrated system through mouth 874 and oropharynx 878 of patient 850 into nasopharynx 884, or nasal cavity 886. As would be appreciated, the oropharynx 878 is the part of the throat just behind mouth 874 that connects mouth 874 to the top of the throat. Again, such an insertion is not easily accomplished with an integrated system.

The unintegrated cryosurgery system 800 illustrated in FIG. 8A provides a system in which the operator may deliver a cryogen treatment to the walls of nasopharynx 884 or nasal cavity 886 by inserting cryogen delivery apparatus 848 through one of nostrils 880. Cryogen delivery apparatus 848, in accordance with the embodiment illustrated in FIG. 8A, comprises cryogen delivery catheter 856, catheter tip 858 having one or more holes 854, and a guide portion 846. As would be appreciated, catheters such as catheter 856, are tubular, flexible, and have at least one lumen disposed along this its elongate body. Using guide portion 846, the operator inserts at least a portion of cryogen delivery catheter 856 through one of nostrils 880 and into nasal cavity 886. Delivery catheter 856 is inserted intro nasal cavity 886 until tip 858 is positioned adjacent the target tissue. As shown in FIG. 8A, delivery catheter 856 is configured to be flexible so as to be curved, shaped, bent, folded etc. In preferred embodiments, delivery catheter 856 may be further constructed from a shape memory material that is capable of retaining the desired curved or bent shape until the shape is altered again by the operator. These properties of delivery catheter 856 assist the operator in achieving the proper placement of tip 858.

As would be appreciated depending on the location of the target tissue (e.g. nasal cavity 886) within patient 850, the operator cannot directly view tip 858, the area adjacent the target tissue, or cryogen delivery apparatus 848 while the cryogen delivery apparatus 848 is in nasal cavity 886. As such, prior to, during, or after insertion of cryogen delivery apparatus 848 into nasal cavity 886, the operator positions insertable visualization device 834 in mouth 874 or oropharynx 878. Where the target tissue is in nasal cavity 886, insertable visualization device 834 is positioned in patient 850 so as to provide the operator with an indirect view of cryogen delivery apparatus 848 tip 858 and/or the target tissue while it is in nasal cavity 886. For example, in the illustrated embodiment, insertable visualization device 834 may comprises comprise a hand-held mirror 834, but may also comprises many other devices providing the same or similar function. For example, insertable visualization device 834 may be an endoscope, a fiber optic cable system, etc. As shown in position shown in FIG. 8A, insertable visualization device 834 is positioned so that the operator is able to view the reflection of tip 858 and/or the target tissue cryogen delivery apparatus 848 in mirror 834.

Although insertable visualization device 834 comprises a hand-held mirror in the embodiment illustrated in FIG. 8A, it should be appreciated that other insertable visualization devices could also be used. For example, insertable visualization device 834 could also comprise an endoscope camera, a fiber optic cable, etc.

After cryogen delivery apparatus 848 and insertable visualization device 834 have been inserted into nasal cavity 886 and into oropharynx 878, respectively, the operator uses foot pedal 810 to start the flow of cryogen as described above with reference to FIG. 7. Cryogen spray, shown as reference 852 in FIG. 8, is emitted from tip 858 to the target tissue. After the target tissue reaches cryofrost, cryogen delivery apparatus 848 is removed from the patient. It should be appreciated that multiple applications of cryogen spray onto target tissue may be provided during treatment. Between each such application, the target tissue may thaw prior to the next application of cryogen.

As noted above, the embodiment of cryogen delivery apparatus 848 illustrated in FIG. 8A further comprises guide portion 846 Guide portion 846 is configured to assist the operator in the insertion and positioning of delivery catheter 856 within patient 850. Guide portion 846 comprises handle 860, support member 862 and release 836. Support member 862 is configured to receive and support delivery catheter 856. In the embodiment illustrated in FIG. 8A, support member 862 member comprises an insulated tubular shape configured to substantially fit around delivery catheter 856. It should be appreciated that support member 862 may take on a variety of other shapes, including concave, half-circular, rectangular, forked, etc. As may appreciated, support member 862 and handle 860 may be longer or shorter depending on the particular needs of the operator.

Positioned on support member 862 is a release 836. Release 836 is configured to retain cryogen delivery catheter 856 in support member 862. Release 836 may comprise, for example, a spring mechanism configured to exert sufficient pressure on delivery catheter 856 when delivery catheter 856 is positioned in support member 862 so as to retain delivery catheter 856 therein. It should be appreciated that release may comprise mechanisms other than a spring mechanism, such as a lever arm or a clip or coupler. In other embodiments, support member 862 is configured to retain cryogen delivery catheter 856 therein via compression or friction. In such embodiments, release 836 is unnecessary.

Although the embodiment illustrated in FIG. 8A discloses cryogen delivery apparatus 848 having guide portion 846 for inserting cryogen delivery catheter 856 into patient 850, it should be appreciated that cryogen delivery apparatus 848 may also be introduced into the patient via a variety of other means, including through the use of a stylet, trocar, sheath, etc. Similarly, it should be appreciated that the present invention is not limited to the embodiment of guide portion 846 illustrated in FIG. 8A. FIG. 8B is a cross-sectional view of the head of a patient 850 having one embodiment of cryosurgery system 582 of FIG. 5B, referred to as cryosurgery system 800, positioned therein. In this illustrative embodiment, cryosurgery system 800 comprises an insertable visualization system 834 and a cryogen delivery apparatus 848.

FIG. 8B illustrates a perspective view of an additional embodiment of unintegrated cryosurgery system 582 of FIG. 5B, referred to as cryosurgery system 800. Similar to FIG. 8A, FIG. 8B is illustrated in conjunction with a simplified cross-sectional view of the head of a patient 850, including nasal cavity 886, frontal sinus 882, sphenoid sinus 880, nasopharynx 884, oropharynx 878, tongue 888, esophagus 876, and palate 892 for reference.

Cryosurgery system 800 of FIG. 8B comprises an insertable visualization device 834 and a cryogen delivery apparatus 848. In this illustrative embodiment, cryosurgery system 800 is configured to provide a cryogen treatment to target tissue, for example, on the walls of esophagus 876. In the embodiment illustrated in FIG. 8B, the operator positions cryogen delivery apparatus 848 into mouth 874 of patient 850. Cryogen delivery apparatus 848 comprises a cryogen delivery catheter 856 and a guide portion 846.

In the embodiment of FIG. 8A, guide portion 846 comprises an insulated tube portion configured to receive cryogen delivery catheter 856. Guide portion 846 is configured to retain cryogen delivery catheter 856. In certain embodiments, guide portion 846 may retain cryogen delivery catheter therein via a friction fit between cryogen catheter 856 and guide portion 846. In other embodiments, guide portion 846 may retain cryogen delivery catheter therein via a compression fit between cryogen catheter 856. In such embodiments, guide portion 846 may comprise a compressible tubular structure having an inside diameter that is less than the outside diameter of at least a portion of catheter 856.

As explained above with reference to FIG. 8A, in certain embodiments, delivery catheter 856 is configured to be flexible so as to be curved, shaped, bent, folded etc. However, in the embodiments shown in FIG. 8B, delivery catheter 856 is not constructed from a shape memory material that is capable of retaining a curved or bent shape for an extended period of time. Instead, guide portion 846 may comprise a resiliently flexible shape memory material. In other embodiments, guide portion 846 comprises a series of interconnected joints which allow guide portion 846 to maintain a curve or bend.

In the illustrated embodiment, after guide portion 846 is formed into a desired shape, delivery catheter 856 is inserted therein. Because guide portion is configured to have sufficient rigidity, and delivery catheter 856 is flexible, delivery catheter 856 will take on the shape of guide portion 846.

In the embodiment shown in FIG. 8B, the operator positions the distal tip of cryogen delivery catheter 856 near the wall of the esophagus 876. As would be appreciated, the operator cannot directly view cryogen delivery apparatus 848 while the cryogen delivery apparatus 848 is in esophagus 876. As such, prior to, during, or after insertion of cryogen delivery apparatus 848 into esophagus 876, the operator positions insertable visualization device 834 in mouth 874 or oropharynx 878 of patient 850. Insertable visualization device 834 is positioned in patient 850 so as to provide the operator with an indirect view of cryogen delivery apparatus 848 while it is in esophagus 876. Insertable visualization device 834 may comprise a hand-held mirror 834, but may also comprise many other devices providing the same or similar function. For example, insertable visualization device 834 may also comprise an endoscope, a fiber optic cable system, etc. As shown in FIG. 8B, insertable visualization device 834 is positioned so that the operator is able to view the target tissue and/or cryogen delivery apparatus 848 in mirror 834.

In the embodiment illustrated in FIG. 8B, cryosurgery system comprises a cryogen storage tank (not shown), a regulation apparatus 816, a control unit 802 and a foot pedal 810. These components are similar to the components described above with reference to FIG. 7. Similar to the embodiment illustrated in FIG. 7, the operator controls the delivery of cryogen spray to the target tissue via foot pedal 810, Although this illustrative embodiment illustrates the use of foot pedal 810, controller 802 and regulation apparatus 816 control the flow of cryogen to cryogen delivery apparatus to provide user inputs to controller 802, it should be appreciated that other manners of entering operator inputs may be utilized, including buttons, switches, toggles, dials, user interfaces, etc. on, in, or coupled to control unit 802.

After the operator positions the distal tip of cryogen delivery catheter 856 near the wall of the esophagus 876, the operator uses foot pedal 810 to start the flow of cryogen. Cryogen spray, shown as reference 852 in FIG. 8B, is emitted from cryogen delivery catheter 856 to the target tissue on the wall of esophagus 876. After the target tissue reaches cryofrost, cryogen delivery apparatus 848 is removed from the patient. It should be appreciated that multiple applications of cryogen spray onto target tissue may be provided during treatment. Between each such application, the target tissue may thaw prior to the next application of cryogen occurs.

For ease of explanation, cryogen delivery apparatus 848 and insertable visualization device 834 have been shown inserted into the mouth of patient 850 to provide cryosurgical treatment to target tissue positioned on the wall of esophagus 876. It should be appreciated that cryogen delivery apparatus 848 and insertable visualization device 834 may be utilized in additional areas or cavities of patient 850, such as the nasal cavity (as shown in FIG. 8A), esophagus, stomach, lung, etc. Similarly, it should be appreciated that in these other embodiments, cryogen delivery apparatus 848 and insertable visualization device 834 may be inserted into patient 850 from different entry points on the body of patient 850. For example, cryogen delivery apparatus 848 may be inserted through the nose of patient 850 while insertable visualization device 834 may be inserted through the mouth of patient 850 to have visual access to separately inserted cryogen delivery apparatus 848. Also, for example, cryogen delivery apparatus 848 may be inserted through a body access interface device such as a trocar while insertable visualization device 834 may be inserted through a separate trocar on the body of patient 850. Other entry points and interfaces now known or later developed may be used in conjunction with other embodiments of the present invention.

It should be appreciated that the cryosurgery system of the present invention is not limited to a single indirect visualization apparatus. For example, the embodiments described above with reference to FIGS. 5A and 5B may be advantageously combined to provide the operator with two or more devices to view the cryogen delivery. Also, two more embodiments of either FIG. 5A or FIG. 8 may be collectively utilized to provide the operator with multiple simultaneous visualization systems or devices. The various combinations of multiple visualization systems available to the user is not limited and the above suggested combinations are merely examples that may be utilized.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

What is claimed is:

1. A cryosurgery system comprising:
a cryogen delivery apparatus configured to deliver a spray of cryogen to target tissue of a patient;
an indirect visualization apparatus configured to provide indirect visualization of the target tissue during the cryogen delivery,
wherein the indirect visualization apparatus and the cryogen delivery apparatus are constructed and arranged to be operationally unintegrated and physically spaced with respect to each other during the delivery of the cryogen.

2. The system of claim 1, wherein said cryosurgery system further comprises:
a cryogen source configured to provide the cryogen;
a regulation apparatus fluidically coupled to the cryogen source and to the cryogen delivery catheter; and
a controller communicatively coupled to the regulation apparatus configured to control the release of cryogen into the cryogen delivery apparatus

3. The system of claim 1, wherein the indirect visualization comprises an external imaging system.

4. The system of claim 3, wherein the external imaging system comprises at least one of the group including:
a x-ray system;
a computed tomography system;
an ultrasound system; and
a magnetic resonance imaging system.

5. The system of claim 1, wherein the indirect visualization comprises:
an insertable visualization device that is separately guidable from the cryogen delivery apparatus.

6. The system of claim 5, wherein the insertable visualization device comprises an endoscope camera.

7. The system of claim 5, wherein the insertable visualization device comprises a handheld mirror.

8. The system of claim 5, wherein the insertable visualization device comprises a fiber optic cable.

9. The system of claim 1, wherein the cryogen delivery apparatus comprises a cryogen delivery catheter.

10. The system of claim 9, wherein the cryogen delivery apparatus further comprises:
a hand tool configured to enable an operator to position the cryogen delivery catheter adjacent the target tissue.

11. The system of claim 10, wherein the hand tool comprises a thermally insulated tube configured to operationally retain the cryogen delivery catheter therein.

12. The system of claim 1, further comprising a suction catheter configured to evacuate the treatment area of the delivered cryogen.

13. The system of claim 1, further comprising at least one additional indirect visualization system.

14. The system of claim 9, wherein the cryogen delivery catheter comprises a tip positioned on the distal end of the catheter, the tip configured to direct the spray of the cryogen to the target tissue.

15. A method of delivering cryogenic to target tissue within a patient via a cryosurgery system, comprising:
adjusting the relative physical orientation of the patient and an indirect visualization apparatus;
inserting a cryogen delivery apparatus that is physically spaced apart from, and operationally unintegrated from the indirect visualization apparatus into the patient;
positioning the cryogen delivery apparatus in the patient without requiring concomitant movement of the indirect visualization apparatus;
delivering the cryogen to the target tissue; and
monitoring the delivery of the cryogen with the physically spaced apart indirect visualization device.

16. The method of claim 15, wherein the indirect visualization apparatus comprises an external imaging system selected from the group comprising:
a x-ray system;
a computed tomography system;
an ultrasound system; and
a magnetic resonance imaging system.

17. The method of claim 15, wherein the indirect visualization apparatus comprises an insertable visualization device selected from the group comprising:
a mirror;
a endoscope camera; and
a fiber optic cable.

18. The method of claim 15 wherein said cryosurgery system further comprises a cryogen source configured to provide the cryogen, a regulation apparatus fluidically coupled to the cryogen source and to the cryogen delivery apparatus, and a controller communicatively coupled to the regulation apparatus and wherein delivering the cryogen comprises:
signaling the regulation apparatus with the controller to release of cryogen into the cryogen delivery apparatus,
regulating the release of cryogen to the apparatus via the regulation apparatus.

19. The method of claim 15, wherein positioning the cryogen delivery catheter comprises:
positioning the cryogen delivery apparatus with a hand tool comprising a thermally insulated tube configured to operationally retain the cryogen delivery apparatus therein.

20. The method of claim 15, further comprising:
suctioning the area of the delivered cryogen using a suction catheter.

21. A cryosurgery system comprising:
a cryogen delivery means for delivering a spray of cryogen to target tissue of a patient;
a viewing means for indirectly viewing the delivery of the cryogen to the target tissue;
wherein the delivery means and the viewing means are configured to be operationally unintegrated and physically spaced with respect to each other during the delivery of the cryogen.

22. The system of claim 21, further comprising:
means for providing the cryogen;
a regulation means fluidically coupled to the means for providing the cryogen and to the delivery means; and
a controller means communicably coupled to the regulation means and configured to control the release of cryogen into the cryogen delivery apparatus via said regulation means.

23. The system of claim 21, wherein the viewing means comprises an external imaging means.

24. The system of claim 23, wherein the external imaging means comprises at least one of the group consisting of:
a x-ray system;
a computed tomography system;
an ultrasound system; and
a magnetic resonance imaging system.

25. The system of claim 21, wherein the viewing means comprises:
an insertable visualization means that is separately guidable from the cryogen delivery means.

26. The system of claim 21, wherein the cryogen delivery means comprises:
a cryogen delivery catheter; and
a hand tool configured to enable an operator to position the cryogen delivery catheter adjacent the target tissue.

27. The system of claim 21, further comprising at least a second viewing means for indirectly viewing the delivery of the cryogen
